(19) United States
(12) Patent Application Publication
(10) Pub. No.: US 2022/0047473 A1
(43) Pub. Date: Feb. 17, 2022

(54) COSMETIC COMPOSITION HAVING CAPSULE

(71) Applicant: KOLMAR KOREA CO., LTD., Sejong-si (KR)

(72) Inventors: Eun Sook JEONG, Gyeonggi-do (KR); Jin Mo KIM, Seoul (KR); In Ki HONG, Seoul (KR)

(21) Appl. No.: 17/392,659

(22) Filed: Aug. 3, 2021

(30) Foreign Application Priority Data
Aug. 13, 2020 (KR) 10-2020-0101918

(51) Int. Cl. A61K 8/11; A61Q 1/08; A61K 8/81; A61K 8/898; A61K 8/34; A61K 8/35

(52) U.S. Cl. CPC A61K 8/11; A61Q 1/08; A61K 8/8147; A61K 8/898; A61K 2800/52; A61K 8/35; A61K 8/345; A61K 2800/43; A61K 8/34

(57) ABSTRACT

A cosmetic composition according to an embodiment of the present disclosure stably contains capsules. In the cosmetic composition according to an embodiment, the capsule where an active ingredient such as a colorant is loaded has excellent physical and chemical stability. Therefore, the cosmetic composition does not have problems of formulation instability which may be caused by contact and reaction between a component in the capsule and a component outside the capsule, even when kept for a long period of time. In addition, the cosmetic composition can obtain a dramatic makeup effect when the colorant loaded in the capsule is discharged only by frictional force generated during use of the cosmetic.

### CROSS REFERENCE TO RELATED APPLICATION

The present application claims priority to Korean Patent Application No. 10-2020-0101918, filed Aug. 13, 2020, the entire contents of which is incorporated herein for all purposes by this reference.

### BACKGROUND

### 1. Field of the Invention

The present invention relates to a cosmetic composition stably containing capsules.

### 2. Description of the Related Art

In the cosmetic field, capsules are mainly used to prevent deterioration of specific ingredients. In the cosmetic field, cosmetics have been developed in which capsules are processed separately from a cosmetic composition and then the capsules are mixed with the cosmetic composition. However, the conventional method is cumbersome and expensive to manufacture. Also, after a cosmetic is applied to the skin, the residue of capsules remains on the skin, causing the user to feel discomfort. Therefore, a technology has been developed in which a capsule manufacturing process is omitted but capsules are automatically formed in cosmetics by utilizing the structure of the skin composed of two primary layers. However, there are problems that the capsules are easily decomposed in the cosmetics, the amount of the cosmetic composition contained in the formed capsules is small, and the capsules vary in size.

Therefore, it is necessary to develop a cosmetic composition containing stable capsules having a constant shape or size even when keeping a cosmetic for a long time.

### SUMMARY

Accordingly, the present invention has been made keeping in mind the above problems occurring in the related art, and an objective of the present invention is to provide a cosmetic composition stably containing capsules.

Another objective of the present invention is to provide a cosmetic composition having a capsule capable of stably carrying an active ingredient.

Still another objective of the present invention is to provide a cosmetic composition having a capsule in which physical and chemical stability is maintained until an external force is applied by a user to use cosmetics.

Still another objective of the present invention is to provide a cosmetic composition having a capsule that is self-encapsulated by a reaction between components in the cosmetic composition.

Still another objective of the present invention is to provide a cosmetic composition having a capsule having excellent dispersibility.

In order to achieve the above objectives, according to one aspect of the present invention, there is provided a cosmetic composition having a capsule, the cosmetic composition including: a capsule where an active ingredient is loaded; and a capsule stabilizer, in which the capsule includes a shell including an anionic polymer and a cationic polymer, and the capsule stabilizer includes a phenyl group-containing alcohol.

In the cosmetic composition according to the present invention, the capsule where an active ingredient such as a colorant is loaded has excellent physical, chemical, and electrical stability. Therefore, the cosmetic composition does not have problems of formulation instability, such as capsule destruction caused by contact and reaction between a component in the capsule and a component outside the capsule, even when kept for a long period of time. In addition, the cosmetic composition can obtain a dramatic makeup effect when the colorant loaded in the capsule is discharged only by frictional force generated during use of the cosmetic.

### DETAILED DESCRIPTION

Hereinafter, the present invention will be described in detail.

Hereinafter, each configuration will be described in more detail, but this is only an example, and the scope of the present invention is not limited by the following content.

The present invention provides a cosmetic composition having a capsule, the cosmetic composition including: a capsule where an active ingredient is loaded; and a capsule stabilizer, wherein the capsule includes a shell including an anionic polymer and a cationic polymer, and the capsule stabilizer includes a phenyl group-containing alcohol.

The capsule may refer to a collector configured in a membrane or shell structure capable of distinguishing internal and external environments and containing a specific component therein.

An active ingredient may be loaded in the capsule. The active ingredient may refer to a substance directly or indirectly involved in obtaining a desired effect of the cosmetic composition. For example, the active ingredient may include a material for improving skin conditions, such as a natural substance extract and a synthetic material, which treats specific diseases such as acne or improves skin conditions. Otherwise, the active ingredient may include a material for imparting an external cosmetic effect such as a colorant and a pigment.

The capsule according to the present invention may be self-encapsulated in a form where a specific component is trapped inside by an action between components in the cosmetic composition without any capsule manufacturing process.

The capsule stabilizer may further include a polyhydric alcohol in addition to the phenyl group-containing alcohol. As the composition includes a phenyl group-containing alcohol or a phenyl group-containing alcohol and a polyhydric alcohol, it is possible to ensure stability in the formulation of the capsule having the shell including the anionic polymer and the cationic polymer. For example, the capsule according to the present invention can minimize the destruction of the capsule shell which may be caused by electrical and chemical interaction between the capsule shell and the component existing in the capsule external environment in the cosmetic composition. Therefore, as the contact is controlled between the components trapped in the capsule and the component existing in the environment outside the capsule, it is possible to effectively control the deterioration of formulation stability such as deposition caused by the reaction between the components.

The capsule stabilizer may be included in a range of 0.3% to 8% by weight based on the total weight of the composition.

Alternatively, the capsule stabilizer may be included in a range of 0.3% to 8% by weight, 0.35% to 7% by weight, 0.4% to 6.5% by weight, or 0.45% to 6% by weight based on of the total weight of the composition. When the content of the capsule stabilizer satisfies the above ranges, excellent capsule stability can be expected. Otherwise, the capsule may be not easily formed, or the formed capsule is easily destroyed.

In case the composition contains a phenyl group-containing alcohol as a capsule stabilizer, the stability of the formed capsule may be improved after capsule formation. Specifically, the phenyl group-containing alcohol of the capsule stabilizer may be included alone without any other capsule stabilizers in a range of 0.3% to 0.8% by weight, based on the total weight of the composition.

The phenyl group-containing alcohol and the polyhydric alcohol may be included as the capsule stabilizer, and in case both the phenyl group-containing alcohol and the polyhydric alcohol are included, the phenyl group-containing alcohol may be included in a range of 0.3% to 0.8% by weight and the polyhydric alcohol may be included in a range of 0.01% to 7.7% by weight, based on the total weight of the composition.

The polyhydric alcohol may include at least one selected from the group consisting of ethylhexylglycerin, 1,2-hexanediol, butylene glycol, propanediol, and dipropylene glycol. Here, based on the total weight of the composition, ethylhexylglycerin, 1,2-hexanediol, butylene glycol, propanediol, and dipropylene glycol may be included in an amount of 0.01% to 0.1% by weight, 0.01% to 2% by weight, 0.01% to 3% by weight, 0.01% to 5% by weight, and 0.01% to 3% by weight.

The composition may further include an appropriate amount of a dispersant, a chelator, a pH adjuster, a solvent, a thickener, a pigment, a film former, a binder, and a functional ingredient such as a moisturizer, a skin conditioning agent, and a functional main ingredient.

The chelator may include disodium ethylenediaminetetraacetic acid (EDTA), trisodium EDTA, or tetrasodium EDTA. The pH adjuster may include tromethamine, triethanolamine, or imidazolidinylurea.

In addition, the functional ingredient may include a moisturizer such as glycerin, a functional cosmetic ingredient having a function such as whitening, wrinkle improvement, and UV protection, an oil thickener such as hectorites and polyurethane-79, or a skin conditioning agent such as octyldodecanol.

In addition, the film former may include a methyl methacrylate cross polymer, silica, or an allyl methacrylate cross polymer.

In addition, the thickener may include an oil thickener such as hectorites, polyurethane-79, and dextrin palmitate.

In addition, the pH adjuster may include tromethamine, triethanolamine, or imidazolidinylurea.

Furthermore, the solvent may include an organic solvent such as hydrogenated poly(C6-14 olefin), diphenyl dimethicone, isononyl isononanoate, dicaprylyl ether, propylene carbonate, dicaprylyl carbonate, isodecyl neopentanoate, and propylheptyl caprylate.

The composition may include: an aqueous phase part including the anionic polymer, the capsule stabilizer, and water; an oil phase part including the cationic polymer; and a powder part including a pigment.

A ratio of the specific gravity of the oil phase part and the aqueous phase part may be in a range of 1:0.5 to 1:1.5. The specific gravity may be measured at 1 atm and 25° C. Preferably, the ratio of the specific gravity of the oil phase part and the aqueous phase part in the composition may be in a range of 1:0.75 to 1:1.25, or specifically, in a range of 1:0.85 to 1:1.20. When the ratio of the specific gravity of the oil phase part and the aqueous phase part in the composition is the same as above, dispersion stability of the capsule formed in the composition can be maximized.

The oil phase part in the composition may be included in an amount of 1% to 95% by weight, based on the total weight of the composition, preferably 10% to 70% by weight, more preferably 15% to 50% by weight.

In addition, the aqueous phase part in the composition may be included in an amount of 10% to 90% by weight based on the total weight of the composition, preferably 20% to 85% by weight, more preferably 60% to 80% by weight.

In one embodiment, the capsule of the cosmetic composition may be self-encapsulated. Specifically, through mixing of the aqueous phase part, the oil phase part, and the powder part, the capsule in a state in which the colorant or the like in the powder part is trapped inside can be produced. Here, based on the total weight of the composition, the aqueous phase part, the oil phase part, and the powder part may be included in an amount of 55% to 83% by weight, 15% to 30% by weight, and 2% to 15% by weight, respectively.

In addition to the anionic polymer, capsule stabilizer and water, the aqueous phase part may further include: a chelator such as disodium EDTA, trisodium EDTA, and tetrasodium EDTA; a moisturizer such as glycerin; a pH adjuster such as tromethamine, triethanolamine, imidazolidinylurea; and a functional main ingredient having a function such as whitening, wrinkle improvement, and UV protection.

In addition to the cationic polymer, the oil phase part may further include: an organic solvent such as hydrogenated poly (C6-14 olefin), diphenyl dimethicone, isononyl isononanoate, dicaprylyl ether, propylene carbonate, dicaprylyl carbonate, isodecyl neopentanoate, and propylheptyl caprylate; hectorites such as disteardimonium hectorite; an oil thickener such as polyurethane-79, dextrin palmitate; and a skin conditioning agent such as octyldodecanol.

In addition, the powder part may include a pigment such as titanium dioxide and iron oxide. The powder part may further include: a film former such as methyl methacrylate cross polymer, silica, and allyl methacrylate cross polymer; a binder such as triethoxycaprylylsilane and silica dimethyl silylate; a surfactant such as disodium stearoyl glutamate; and a coating agent such as aluminum hydroxide or aluminum dimyristate.

In addition, assuming the cationic polymer is 100 parts by weight, the anionic polymer may be included in an amount of 10 part by weight to 150 parts by weight, preferably 15 parts by weight to 120 parts by weight, more preferably 20 parts by weight to 100 parts by weight, still more preferably parts by weight to 90 parts by weight, still more preferably 40 parts by weight to 80 parts by weight. If the content of the anionic polymer in the composition is out of the above ranges, formulation stability may be deteriorated, for example, discoloration and viscosity change. In addition, the stability of the capsule may be deteriorated, for example, destruction of the capsule.

The composition may be an oil-in-water composition or a water-in-oil composition.

A diameter of the capsule of the composition may be in a range of 0.01 micrometers to 5000 micrometers, or specifically 0.1 micrometers to 3000 micrometers, or further specifically 1 micrometer to 2000 micrometers, or still further specifically 50 micrometers to 1000 micrometers. If the diameter of the capsule is out of the above ranges, the capsule may be easily destroyed, or the amount of active ingredient loaded therein may be severely small.

In addition, the anionic polymer may include at least one selected from the group consisting of carbomer, sodium carbomer, sodium acrylate crosspolymer-2, hydroxyethyl acrylate, and sodium acryloyldimethyltaurate copolymer, and at least one selected from the group consisting of ammonium acryloyldimethyltaurate/VP copolymer, sodium polyacryloyldimethyl taurate, ammonium acryloyldimethyltaurate/beheneth-25 methacrylate crosspolymer and acrylates/C₁₀₋₃₀ alkyl acrylate crosspolymer, and anionic polysaccharide.

Preferably, the anionic polymer may include a combination of carbomer and acrylates/C₁₀₋₃₀ alkyl acrylate crosspolymer.

In addition, the cationic polymer may include at least one of aminopropyl dimethicone and amodimethicone, preferably amodimethicone.

In addition, a viscosity of the cosmetic composition may be in a range of 3,000 cps to 20,000 cps, or specifically 5,000 cps to 18,000 cps, or further specifically 6,000 cps to 15,000 cps.

In addition, the formulation of the cosmetic may be a gel type.

The cosmetic composition may include a foundation, tone-up cosmetics, a sunscreen, an essence, a primer, a concealer, and the like.

Hereinafter, the present invention will be described in more detail with reference to examples. It should be apparent to those skilled in the art that these examples are for illustrating the present invention only and that the scope of the present invention is not construed as being limited by these examples.

### [Preparation Example] Preparation of Foundation

A foundation was prepared using ingredients shown in Table 1 below. Specifically, an aqueous phase part was prepared by mixing Components 1 to 5 of Table 1 below except for Component 3. Then, the Component 3 was weighed, mixed with 4% water, put into the aqueous phase, and mixed. In addition, Components 6 to 8 were mixed to prepare an oil phase part. Furthermore, a powder part obtained by mixing and homogenizing Components 9 to 12 was put into the oil phase part and mixed. Finally, while stirring the aqueous phase with an AGI homo mixer, a mixture of the powder part and the oil phase part were added thereto to prepare a cosmetic composition.

TABLE 1

| | | | Content |
|---|---|---|---|
| No. | Phase | Component | (wt. %) |
| 1 | Aqueous | water | To 100 |
| 2 | phase | carbomer | 0.30 |
| 3 | | tromethamine | 0.24 |
| 4 | | phenoxyethanol | 0.20 |
| 5 | | 1,2-hexanediol | 2.0 |
| 6 | Oil | hydrogenated polydecene | 20 |
| 7 | phase | amodimethicone | 0.30 |
| 8 | | disteardimonium hectorite | 0.1 |
| 9 | Powder | titanium dioxide | 9.6 |
| 10 | phase | iron dioxide (CI 77492; | 0.3 |
| | | hydrated ferric oxide; yellow | |
| | | oxide of iron; iron oxides) | |
| 11 | | iron dioxide (CI 77491; ferric | 0.099 |
| | | oxide; red oxide of iron; iron oxides) | |
| 12 | | iron dioxide (CI 77499; | 0.001 |
| | | ferrous-ferric oxide; iron oxides) | |

### [Experimental Example 1] Capsule Formation and Stability Evaluation According to Types and Content of Capsule Stabilizer

As shown in Table 2 below, highly adhesive capsule foundations were prepared by varying the content of the components. Then, whether capsules were formed and whether capsules were stable were checked.

Capsule stability was evaluated by measuring and visually observing shapes and sizes of the capsules over time in a chamber at a cycle of 5° C., room temperature, 40° C., and 50° C.

Components not mentioned in Table 2 compared to those of Table 1 were used under the same conditions as in Table 1. In Examples 1 to 4 and Comparative Examples 1 to 7, the ratio of the oil phase to the aqueous phase was 1:0.88.

TABLE 2

| | | | | | | Com- | Com- | Com- | Com- | Com- | Com- | Com- |
|---|---|---|---|---|---|---|---|---|---|---|---|---|
| | | | | | | para- | para- | para- | para- | para- | para- | para- |
| | | | | | | tive | tive | tive | tive | tive | tive | tive |
| | | Exam- | Exam- | Exam- | Exam- | Exam- | Exam- | Exam- | Exam- | Exam- | Exam- | Exam- |
| Component | | ple 1 | ple 2 | ple 3 | ple 4 | ple 1 | ple 2 | ple 3 | ple 4 | ple 5 | ple 6 | ple 7 |
| Anionic | carbomer | 0.21 | 0.21 | 0.28 | 0.3 | 0.26 | 0.26 | 0.26 | 0.26 | 0.26 | 0.26 | 0.26 |
| polymer | | | | | | | | | | | | |
| Cationic | amodimethicone | 0.3 | 0.3 | 0.3 | 0.3 | 0.3 | 0.3 | 0.3 | 0.3 | 0.3 | 0.3 | 0.3 |
| polymer | | | | | | | | | | | | |
| Capsule | phenoxyethanol | 0.3 | 0.4 | 0.5 | — | — | — | 0.4 | 0.9 | 0.2 | 0.4 | 0.4 |
| stabilizer | | | | | | | | | | | | |
| | hydroxyacetophenone | — | — | — | 0.5 | — | — | — | — | — | — | — |
| | ethylhexylglycerin | — | 0.05 | 0.06 | 0.05 | — | — | 0.05 | — | — | 0.05 | — |
| | 1,2-hexanediol | — | 1.5 | 1 | 1 | — | — | 1 | — | — | 1.5 | — |
| | butylene glycol | — | — | 2 | — | — | — | — | — | — | 3.5 | — |
| | dipropylene glycol | — | 2 | 2 | — | — | — | — | | — | 2 | — |
| | propanediol | — | 1 | 1 | — | — | — | — | — | | 1 | — |
| | pentylene glycol | — | | | | — | — | — | — | — | — | 3 |
| | dehydroacetic acid/ | — | — | — | | — | 0.5 | 0.3 | — | — | — | — |
| | benzyl alcohol | | | | | | | | | | | |
| | sodium benzoate | — | — | — | | 0.3 | — | — | — | — | — | — |

TABLE 3

| | | | | | Com- | Com- | Com- | Com- | Com- | Com- | Com- |
|---|---|---|---|---|---|---|---|---|---|---|---|
| | | | | | para- | para- | para- | para- | para- | para- | para- |
| | | | | | tive | tive | tive | tive | tive | tive | tive |
| | Exam- | Exam- | Exam- | Exam- | Exam- | Exam- | Exam- | Exam- | Exam- | Exam- | Exam- |
| | ple 1 | ple 2 | ple 3 | ple 4 | ple 1 | ple 2 | ple 3 | ple 4 | ple 5 | ple 6 | ple 7 |
| Capsule | Stable | Stable | Stable | Stable | Unstable | Unstable | Unstable | Unstable | Unstable | Unstable | Unstable |
| stability | | | | | | | | | | | |

As a result, when the capsule stabilizer was included in a total amount of 0.3% to 8.0% by weight and the phenyl group-containing alcohol was included as a capsule stabilizer in a range of 0.3% to 0.8% by weight, it was observed that capsules were stably formed in the cosmetic.

When the dehydroacetic acid/benzyl alcohol or sodium benzoate was included, as the capsule stabilizer, instead of or in addition to the phenyl group-containing alcohol, or when the phenyl group-containing alcohol was included, as the capsule stabilizer, in an amount that is out of the above content range, it was observed that the stability of the formed capsules was greatly deteriorated.

### [Experimental Example 2] Evaluation of Dispersion of Capsules According to Specific Gravity Ratio of Oil Phase and Aqueous Phase

Examples 5 to 10 were prepared in the same composition as Example 3, except that the ratio of the oil phase and water phase was changed as follows by adjusting the content of the powder phase dispersed in the oil phase within a range of 5% by weight. Then, dispersion of capsules in cosmetics was evaluated.

Dispersion was evaluated by observing the dispersion stability of capsules in cosmetics in a chamber at a cycle of 5° C., room temperature, 40° C., and 50° C.

TABLE 4

| | Example | Example | Example | Example | Example | Example |
|---|---|---|---|---|---|---|
| | 5 | 6 | 7 | 8 | 9 | 10 |
| Ratio of | 1:0.48 | 1:0.75 | 1:0.97 | 1:1.13 | 1:1.25 | 1:1.6 |
| specific | | | | | | |
| gravity | | | | | | |
| (Oil | | | | | | |
| phase: | | | | | | |
| Aqueous | | | | | | |
| phase) | | | | | | |
| Capsule | X | Δ | ◯ | ◯ | Δ | X |
| dispersion | | | | | | |
| stability | | | | | | |

◯: All formed capsules were stably and evenly dispersed
Δ: Some capsules were skewed upwards or downwards and dispersed
X: All the formed capsules were skewed upwards or downwards and dispersed

As a result, when the ratio of the specific gravity of the oil phase and the aqueous phase was in a range of 1:0.5 to 1:1.5, excellent dispersibility was generally exhibited.

Otherwise, the dispersion was poor, for example, capsules dispersed in the upper or lower side of the cosmetic.

Especially when the ratio of oil phase:water phase was in a range of 1:0.85 to 1:1.2, all capsules were stably and evenly distributed, resulting in excellent aesthetics.

### [Experimental Example 3] Formulation Change Over Long Time According to the Ratio of Anionic Polymer and Cationic Polymer

Examples 11 to 15 were prepared in the same composition as in Example 3, except that the weights of the anionic polymer and the cationic polymer were changed as follows. Then, the formulation change of the cosmetic composition according to the ratio of anionic polymer and cationic polymer was observed over long time.

Formulation change over long time was evaluated by visually observing or measuring pH change, discoloration, viscosity change, capsule precipitation, etc. of the product in a chamber at a cycle of 5° C., room temperature, 40° C., and 50° C.

TABLE 5

| | | Example | Example | Example | Example | Example |
|---|---|---|---|---|---|---|
| Component | | 11 | 12 | 13 | 14 | 15 |
| Anionic | carbomer | 0.1 | 0.18 | 0.21 | 0.26 | 0.28 |
| polymer | | | | | | |
| Cationic | amodime | 0.3 | 0.3 | 0.3 | 0.3 | 0.3 |
| polymer | thicone | | | | | |

TABLE 6

| | Example | Example | Example | Example | Example |
|---|---|---|---|---|---|
| | 11 | 12 | 13 | 14 | 15 |
| Formulation | x | ∘ | ∘ | Δ | x |
| change over | | | | | |
| long time | | | | | |

∘: No change even after 6 months
Δ: Partial degenerated after 3 months
x: Mostly degenerated within 1 month

As a result, it was observed the formulation stability was the best when the anionic polymer was included in an amount in a range of 40 part by weight to 80 parts by weight based on 100 parts by weight of the cationic polymer.

What is claimed:

1. A cosmetic composition comprising:
a capsule where an active ingredient is loaded, the capsule comprising a shell made from an anionic polymer and a cationic polymer; and
a capsule stabilizer comprising a phenyl group-containing alcohol.

2. The cosmetic composition of claim 1, wherein the capsule stabilizer further comprises a polyhydric alcohol.

3. The cosmetic composition of claim 1, wherein the capsule stabilizer is included in an amount of 0.3% to 8.0% by weight based on the total weight of the composition.

4. The cosmetic composition of claim 1, wherein the active ingredient comprises at least one selected from among a pigment and a material for improving skin conditions.

5. The cosmetic composition of claim 1, wherein the cosmetic composition comprising:
an aqueous phase part including the anionic polymer, the capsule stabilizer, and water;
an oil phase part including the cationic polymer; and
a powder part including a pigment.

6. The cosmetic composition of claim 5, wherein a ratio of specific gravity of the oil phase part and the aqueous phase part is in a range of 1:0.5 to 1:1.5.

7. The cosmetic composition of claim 1, wherein the anionic polymer is included in an amount of 10 parts by weight to 150 parts by weight based on 100 parts by weight of the cationic polymer.

8. The cosmetic composition of claim 1, wherein the anionic polymer comprises at least one selected from among carbomer and acrylates/C₁₀₋₃₀ alkyl acrylate crosspolymer;
the cationic polymer comprises amodimethicone or aminopropyl dimethicone; and
the phenyl group-containing alcohol comprises phenoxyethanol or hydroxyacetophenone.

9. The cosmetic composition of claim 2, wherein the polyhydric alcohol comprises at least one selected from the group consisting of ethylhexylglycerin, 1,2-hexanediol, butylene glycol, propanediol, and dipropylene glycol.

10. The cosmetic composition of claim 1, wherein the cosmetic composition comprising the phenyl group-containing alcohol and a polyhydric alcohol as the capsule stabilizer; and
the phenyl group-containing alcohol is included in an amount of 0.3% to 0.8% by weight, and the polyhydric alcohol is included in an amount of 0.01% to 7.7% by weight, based on the total weight of the composition.
